(19) United States
(12) Patent Application Publication
(10) Pub. No.: US 2021/0393540 A1
(43) Pub. Date: Dec. 23, 2021

(54) NANOPARTICLE-ENCAPSULATED CANNABINOIDS AND METHODS FOR MAKING AND USING SAME

(71) Applicant: NuRevelation, LLC, Durham, NC (US)

(72) Inventors: Naomie Lucas, Durham, NC (US); Eric Seidel, Durham, NC (US)

(21) Appl. No.: 17/351,337

(22) Filed: Jun. 18, 2021

Related U.S. Application Data
(60) Provisional application No. 63/158,757, filed on Mar. 9, 2021.
(60) Provisional application No. 63/041,544, filed on Jun. 19, 2020.

(51) Int. Cl. A61K 9/51; A61K 31/05; A61K 31/352

(52) U.S. Cl. CPC A61K 9/5192; A61K 9/5123; B82Y 5/00; A61K 31/352; A61K 31/05

(57) ABSTRACT

Provided are water-soluble and/or alcohol-soluble cannabinoid nanoparticles recovered from a dispersion comprising a sugar, sugar alcohol, or sugar substitute; water; and a cannabinoid, the dispersion comprising less than about 10% by weight of alcohol. Also provided are products comprising the cannabinoid nanoparticle prepared by the disclosed method, including a variety of comestible products.

### CROSS-REFERENCE TO RELATED APPLICATIONS

This application claims priority to U.S. Provisional Application No. 63/041,544, filed Jun. 19, 2020, and U.S. Provisional Application No. 63/158,757, filed Mar. 9, 2021, both of which are incorporated by reference in their entirety.

### BACKGROUND

Demand is increasing for foods, beverages, supplements, and other comestible products that contain cannabinoids, such as cannabidiol (CBD), for example. Of particular interest are water-based or alcohol-based products, such as beverages and water-based comestibles that include one or more cannabinoids, in addition to solid or semi-solid comestibles. Unfortunately, the majority of cannabinoids, including CBD, are hydrophobic, and therefore have limited solubility in water. Although progress has been made in producing water-soluble or alcohol-soluble cannabinoid products, current practices have several significant drawbacks.

For example, because many cannabinoids have undesirable flavors, it is useful to develop a method that masks any bitter aftertaste and other taste properties of cannabinoids in foods, beverages, supplements, and other comestible products. However, current methods for incorporating cannabinoids into such products fail to adequately address the problem of cannabinoid taste. Current methods for incorporating cannabinoids into comestible products also have drawbacks with respect to the manufacturing process. For example, known methods generally require high volumes of additives to increase cannabinoid solubility, which can dilute the concentration of the active cannabinoid in the product.

Similarly, some methods for producing a water-soluble or alcohol-soluble cannabinoid product require the use of organic solvents to solubilize the cannabinoid, including for example, hexane, ethanol, and isopropyl alcohol. Other methods require the use of oils to encapsulate the cannabinoid, such as a carrier oil suitable for forming an oil-in-water emulsion. Such substances can be undesirable in comestible products for a variety of reasons, including toxicity and alteration of taste. In addition, known methods can also alter the cannabinoid itself, which can result in the formation of unknown or undesirable byproducts.

It is therefore desirable to develop alternatives to existing techniques for incorporating cannabinoids into comestible products, including foods and beverages. Particularly, a need exists for a process capable for producing a water-soluble and/or alcohol-soluble cannabinoid nanoparticle that is efficient, scalable, and which results in a product with a suitable concentration of the cannabinoid ingredient. It is also desirable to develop a water-soluble or alcohol-soluble cannabinoid composition that is edible, that masks the taste of the cannabinoid, and that can easily be introduced into downstream product formulations. These needs and others are met by the following methods and products.

### SUMMARY

In one aspect, this disclosure relates to methods for making a cannabinoid nanoparticle, comprising recovering the nanoparticle from a dispersion comprising a sugar, sugar alcohol, or sugar substitute, water, and a cannabinoid, wherein the mass ratio of the cannabinoid to the sugar, sugar alcohol, or sugar substitute is from about 1:300 to about 1:5, and the dispersion comprises less than 10% by weight of alcohol.

In a further aspect, the disclosure relates to a product comprising the cannabinoid nanoparticle prepared by a disclosed method. In one aspect, the product can be a water-based or alcohol-based comestible comprising the cannabinoid nanoparticle prepared by a disclosed method. In another aspect, the product can be a solid or semi-solid comestible comprising the cannabinoid nanoparticle prepared by a disclosed method, together with one or more foodstuff ingredients.

In a further aspect, disclosed is a method of treating a condition in a subject, comprising administering to the subject the cannabinoid nanoparticle prepared by a disclosed method. In a still further aspect, disclosed is a method of treating a condition in a subject, comprising administering to the subject the product prepared by a disclosed method, a disclosed water-based or alcohol-based comestible, or a disclosed solid or semi-solid comestible.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawing, which is incorporated in and constitutes part of this specification and together with the description, serves to explain the principles of the disclosure.

FIG. 1 is a process flow diagram illustrating an exemplary aspect of making a disclosed cannabinoid nanoparticle.

FIG. 2 is a Transmission Electron Microscopy (TEM) image of an exemplary nanoparticle-encapsulated CBD sample prepared according to the disclosed methods.

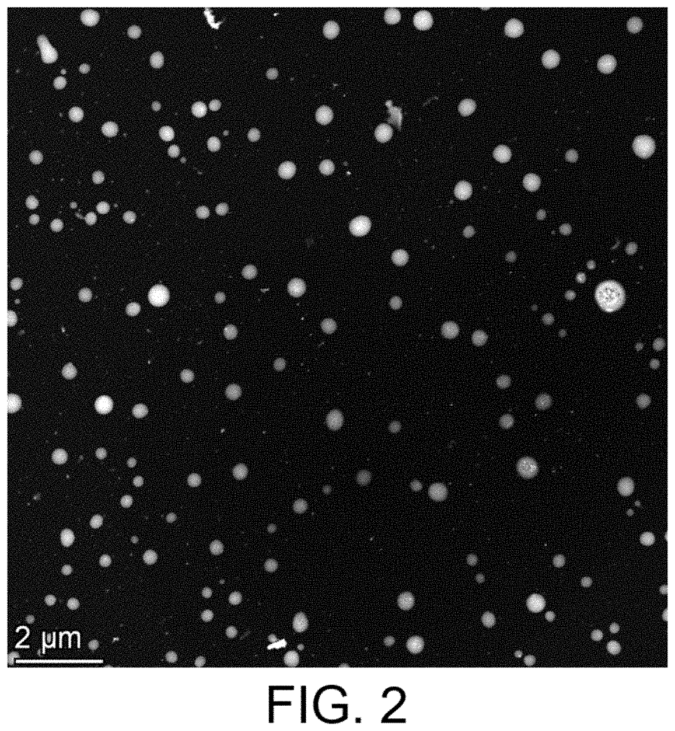

FIG. 3 shows plots of NMR spectra demonstrating the stability of CBD after encapsulation into nanoparticles using the disclosed methods. As shown, CBD remains intact after encapsulation (bottom spectrum) relative to a sample non-encapsulated CBD sample (top spectrum).

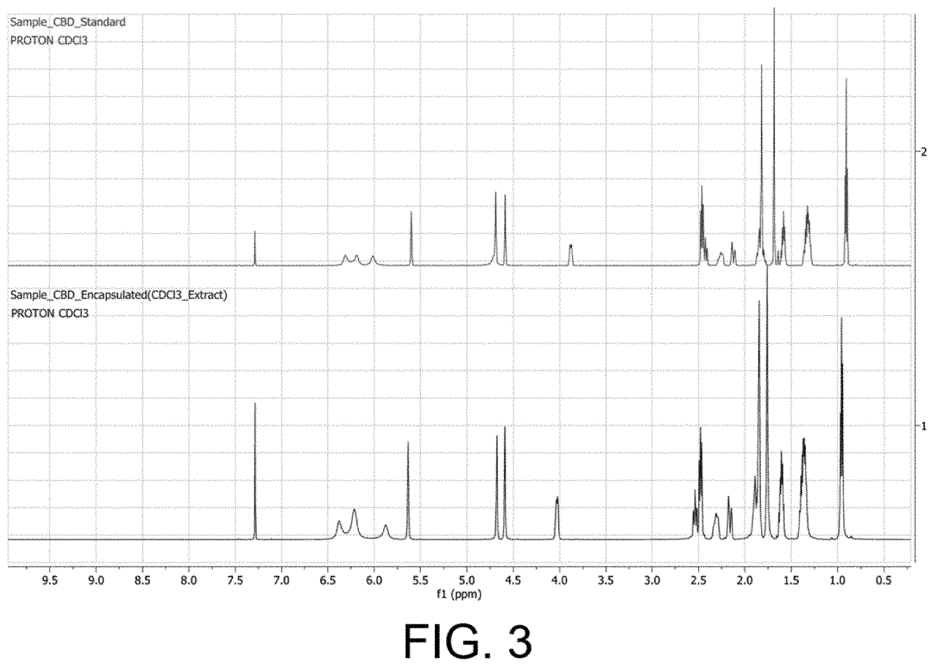

### DETAILED DESCRIPTION

The present invention can be understood more readily by reference to the following detailed description of the invention and the Examples included therein.

Disclosed are components that can be used to perform the disclosed methods. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and products. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

While aspects of this disclosure can be described and claimed in a particular statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present application is not entitled to antedate such publication by virtue of prior invention. Further, stated publication dates may be different from actual publication dates, which can require independent confirmation.

### A. Definitions

Listed below are definitions of various terms. These definitions apply to the terms as they are used throughout this specification, unless otherwise limited in specific instances, either individually or as part of a larger group.

As used in the specification and in the claims, the term “comprising” can include the aspects “consisting of” and “consisting essentially of”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

References in the specification and concluding claims to parts by weight of a particular element or component in a composition or product, denotes the weight relationship between the element or component and any other elements or components in the composition or product for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

The terms “fruit water,” and “plant water,” as used herein, refer to the liquid fluid or juice that can be derived from the fruit, plant, or a vegetable produced by the plant. Non-limiting examples of fruit waters include coconut water, pineapple water, cherry water, mango water, apple water, pomegranate water, and the like. Non-limiting examples of plant or vegetable waters include cactus water, aloe vera water, beet water, carrot water, and the like.

As used herein, the term “sugar” is a collective term encompassing a variety of monosaccharides (e.g., glucose, dextrose, fructose, galactose), disaccharides (e.g., sucrose, lactose, maltose, trehalose), and oligosaccharides or polysaccharides (e.g., maltodextrin).

The term “sugar alcohol,” as used herein, refers to organic compounds, typically derived from a sugar, containing one hydroxyl group attached to each carbon atom. Sugar alcohols are also known as polyhydric alcohols, polyalcohols, alditols or glycitols.

As used herein, the term “sugar substitute” refers to a food additive that provides a sweet taste similar to that of sugar while containing less food energy than sugar-based sweeteners, e.g., a zero-calorie or low-calorie sugar substitute. Suitable sugar substitutes include those that do not include sucrose, fructose, or glucose, for example. The sugar substitute can be natural (e.g., plant derived) or artificial.

“Stevia,” as used herein, refers to any product derived from Stevia rebaudiana including the leaves thereof, and any product comprising steviol glycosides. Examples include without limitation liquid or solid stevia sweeteners, e.g., stevia extract, stevia leaf extract, stevia powder, stevia extract powder, organic stevia, sugar-free stevia, and the like.

The term “cannabinoid,” as used herein, refers to a class of chemical compounds capable of interacting with any mammalian cannabinoid receptor, for example the human CB₁ or CB₂ receptor. The term encompasses naturally-occurring cannabinoids (e.g., phytocannabinoids found in the cannabis plant), synthetic cannabinoids, cannabinoid mimetics, as well as salts, precursors, and metabolites thereof.

“Nanoparticle,” as used herein, refers to a solid nanoparticle entity formed by physical aggregation or noncovalent chemical association (e.g., through one or more noncovalent bonds) of two or more molecular entities, e.g., a cannabinoid and a sugar, sugar alcohol, or sugar substitute. In one aspect, a disclosed cannabinoid is encapsulated within the nanoparticle. “Cannabinoid nanoparticle,” as used herein, refers to a solid nanoparticle encapsulating a cannabinoid with a sugar, sugar alcohol, or sugar substitute carrier, and includes for example, nanoparticles in which the cannabinoid is at least partially encapsulated by the sugar, sugar alcohol, or sugar substitute. “Cannabinoid nanoparticle” also includes nanoparticles in which the cannabinoid and sugar, sugar alcohol, or sugar substitute is self-assembled through physical aggregation or noncovalent chemical association, in addition to nanoparticles that have a micelle or micelle-like structure. In general, the disclosed nanoparticles have a size ranging from about 200 nm to about 2,000 nm, e.g., from about 200 nm to about 1,000 nm, or from about 200 nm to about 500 nm. Particle size can be determined using methods known in the art, e.g., light scattering or zeta potential measurements. Particle size, as referred to herein, refers to the mean or average particle size of a given cannabinoid nanoparticle sample.

The term “mass ratio,” as used herein, refers to the mass of one substance (S1) relative to the mass of another substance (S2), where both masses have identical units (e.g., grams), expressed as S1:S2. For a substance such as water with a density of about 1 mg/mL, it is understood that reference to a volume of water (e.g., in mL) is equivalent to mass (e.g., in units of mg).

As used herein, the term “comestible” refers to any edible product or product suitable for ingestion by a human or animal. The term includes without limitation a liquid comestible (e.g., a water-based or alcohol-based product), and a solid or semi-solid comestible, including various food products, supplements, vitamins, ingestible therapeutic products, and the like.

As used herein, the term “subject” can be a vertebrate, a mammal, a fish, a bird, a reptile, an amphibian, or an invertebrate. Thus, the subject of the herein disclosed methods can be a human, non-human primate, horse, pig, rabbit, dog, sheep, goat, cow, cat, guinea pig or rodent. The subject can also be non-mammalian, e.g., a parakeet or a zebrafish. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the term “subject” includes all categories of food-producing animals (e.g., livestock, poultry, and aquaculture species), companion animals (e.g., dogs, cats, horses, guinea pigs), laboratory animals, and animals maintained in zoological parks.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a clinical, medical, or physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A temperature or temperature range, as expressed herein, refers to the temperature or temperature range at a pressure of 1 atm and equivalents thereof. For example, the phrase “at a temperature equivalent to from about 210° F. to about 280° F. at a pressure of 1 atm” refers not only to the temperature range at the stated atmospheric pressure but also to equivalent temperatures at lower and higher atmospheric pressures. Thus, a stated temperature range can encompass a lower equivalent temperature range at a pressure lower than 1 atm and a higher equivalent temperature range at a pressure higher than 1 atm. Similarly, in some aspects, a stated temperature range can encompass a higher equivalent temperature range at a pressure lower than 1 atm to a achieve a kinetic energy equivalent to that achieved at the stated temperature range.

### B. Methods for Making the Cannabinoid Nanoparticle

In one aspect, the disclosed cannabinoid nanoparticle can be made by recovering the nanoparticle from a dispersion comprising a sugar, sugar alcohol, or sugar substitute, water, and a cannabinoid, wherein the mass ratio of the cannabinoid to the sugar, sugar alcohol, or sugar substitute is from about 1:300 to about 1:5, and the dispersion comprises less than 10% by weight of alcohol.

1. Preparing the Dispersion

In various aspects, the dispersion can be prepared by mixing the desired amount of sugar, sugar alcohol, or sugar substitute, water, and cannabinoid. In one aspect, the sugar, sugar alcohol, or sugar substitute can first be mixed with water, followed by addition of the desired amount of cannabinoid. In one aspect, the water can be distilled, filtered, or otherwise purified to remove impurities typically present in tap water.

In one aspect, the water used for preparing the dispersion can distilled, filtered, or otherwise purified and have a suitable pH. In a further aspect, the purified water used to prepare the dispersion can have a pH of from about 6 to about 9. In a still further aspect, the purified water used to prepare the dispersion can have a pH of from about 7 to about 8. In yet a further aspect, the purified water used to prepare the dispersion can have a pH within or near physiological limits, i.e., about 7 to about 8, or about 7.2 to about 7.5.

In some aspects, the water used for preparing the dispersion can be fruit or plant water, including water derived from a vegetable produced from a plant. Non-limiting examples of suitable fruit waters include coconut water, pineapple water, cherry water, mango water, apple water, pomegranate water, and the like. Non-limiting examples of suitable plant or vegetable waters include cactus water, aloe vera water, beet water, carrot water, and the like. In some aspects, the fruit or plant water can comprise from about 70% to about 98% water by weight. In a further aspect, the fruit or plant water comprising from about 70% to about 98% water by weight can be further diluted with additional water.

In one aspect, the dispersion can be prepared in a vessel comprising a surface that will not adhere to the cannabinoid, e.g., stainless steel. The inventors have discovered that the use of vessels comprising Teflon and silicone results in the cannabinoid adhering to the Teflon or silicone. Similarly, in various aspects, any utensils used for stirring, mixing, or agitating the dispersion can be stainless steel or glass, as the inventors have discovered that the use of wooden utensils can result in the cannabinoid sticking to the wood.

In various aspects, the mass ratio of the sugar, sugar alcohol, or sugar substitute to the water can vary generally depending on the amount of cannabinoid desired. In one aspect, for example, the mass ratio of the sugar, sugar alcohol, or sugar substitute to the water is from about 1:8 to about 1:1.2 prior to the recovering step, i.e., before the volume of the dispersion is reduced during any heating step. In a further aspect, the mass ratio of the sugar, sugar alcohol, or sugar substitute to the water is from about 1:8 to about 1:1.3 prior to the recovering step. In a still further aspect, the mass ratio of the sugar, sugar alcohol, or sugar substitute to the water is from about 1:8 to about 1:1.5 prior to the recovering step. In yet a further aspect, the mass ratio of the sugar, sugar alcohol, or sugar substitute to the water can be about 1:8, 1:7, 1:6, 1:5, 1:4, 1:3, 1:2, 1:1.5, 1:1.3, or 1:1.2 prior to recovering the cannabinoid nanoparticle from the dispersion. In some aspects, the mass ratio of the sugar, sugar alcohol, or sugar substitute to the water is from about 1:6 to about 1:4 prior to the recovering step. In one aspect, the mass ratio of the sugar, sugar alcohol, or sugar substitute to the water is from about 1:4 prior to the recovering step.

In one aspect, for example, it is contemplated that when the amount of cannabinoid added to the dispersion ranges from about 2-6 grams, the mass ratio of the sugar, sugar alcohol, or sugar substitute to the water can be about 3:4 (or about 1:1.33) or less. In a further aspect, when the amount of cannabinoid added to the dispersion is 6 grams or above (e.g., 6-12 grams), the mass ratio of the sugar, sugar alcohol, or sugar substitute to the water can be at least about 1:4. In other words, according to some aspects, more water can be added to the dispersion as the amount of cannabinoid added to the dispersion increases. Amounts of the cannabinoid and other components of the dispersion can be scaled up as desired.

The mass ratio of the cannabinoid to the sugar, sugar alcohol, or sugar substitute in the dispersion can vary. In one aspect, the mass ratio of the cannabinoid to the sugar, sugar alcohol, or sugar substitute is from about 1:300 to about 1:5. In another aspect, the mass ratio of the cannabinoid to the sugar, sugar alcohol, or sugar substitute is from about 1:50 to about 1:5. In a further aspect, the mass ratio of the cannabinoid to the sugar, sugar alcohol, or sugar substitute is from about 1:50 to about 1:10. In another aspect, the mass ratio of the cannabinoid to the sugar, sugar alcohol, or sugar substitute is from about 1:40 to about 1:10. In a further aspect, the mass ratio of the cannabinoid to the sugar, sugar alcohol, or sugar substitute is from about 1:30 to about 1:10. In a still further aspect, the mass ratio of the cannabinoid to the sugar, sugar alcohol, or sugar substitute is from about 1:30 to about 1:15. For example, the mass ratio of the cannabinoid to the sugar, sugar alcohol, or sugar substitute can be about 1:50, 1:45, 1:40, 1:35, 1:30, 1:25, 1:20, 1:18, 1:16, 1:15, 1:13, 1:12, 1:11, 1:10, 1:9, 1:8, 1:7, or 1:5. In one aspect, the mass ratio of the cannabinoid to the sugar, sugar alcohol, or sugar substitute is about 1:20.

2. Recovering the Cannabinoid Nanoparticle

In one aspect, the dispersion comprising the sugar, sugar alcohol, or sugar substitute, water, and cannabinoid can be heated at a temperature equivalent to from about 210° F. to about 280° F. at a pressure of 1 atm for a sufficient time to reduce the volume of the dispersion. As the volume of the dispersion decreases, the cannabinoid nanoparticle can solidify from the dispersion, e.g., by precipitation or coprecipitation of the cannabinoid and the sugar, sugar alcohol, or sugar substitute, thereby forming the cannabinoid nanoparticle. In a further aspect, the dispersion comprising the sugar, sugar alcohol, or sugar substitute, water, and cannabinoid can be heated at a temperature equivalent to from about 220° F. to about 240° F. at a pressure of 1 atm. In a still further aspect, the dispersion comprising the sugar, sugar alcohol, or sugar substitute, water, and cannabinoid can be heated at a temperature equivalent to from about 225° F. to about 230° F. at a pressure of 1 atm. In yet another aspect, the dispersion comprising the sugar, sugar alcohol, or sugar substitute, water, and cannabinoid can be heated at a temperature equivalent to a temperature at a pressure of 1 atm sufficient to induce the dispersion to boil for a sufficient time to reduce the volume of the dispersion and thereby solidify the cannabinoid nanoparticle.

In some aspects, when the dispersion is heated to a temperature sufficient to induce the dispersion to boil for a sufficient amount of time at a stated pressure, equivalent boiling temperatures at different pressures can be calculated according to the Clausius-Clapeyron equation:

$$ln ( {P}_{2} ) - ln ( {P}_{1} ) = \frac{Δ {H}_{v a p}}{R} [ \frac{1}{{T}_{1}} - \frac{1}{{T}_{2}} ]$$

where P₁ and T₁ are standard atmospheric pressure and the known boiling point of water, respectively, ΔHᵥₐₚ is the enthalpy of vaporization of water, and R is the gas constant (8.3145 J/mol*K). Using the Clausius-Clapeyron equation, for example, it can be determined that the boiling temperature of water at 2 atm of pressure (about 247° F.) can be equivalent to the boiling temperature of water at 1 atm of pressure (about 212° F.). Other known methods for determining temperatures equivalent to stated temperatures at stated atmospheric pressures can also be used.

The dispersion can be heated to the desired temperature or temperature range using methods known in the art. In one aspect, for example, the dispersion can be heated in a suitable vessel (e.g., a stainless steel vessel) by a suitable heat source, such as, for example, an induction cooktop. The heat source such as an induction cooktop can be maintained at a suitable temperature or temperature range such that the temperature of the dispersion stays at a temperature equivalent to from about 210° F. to about 280° F. at a pressure of 1 atm. Temperature of the dispersion can be monitored during the heating step using a thermometer, thermocouple, or other suitable device. The dispersion can also be heated under pressure at a suitable temperature equivalent to those described herein.

In various aspects, once the dispersion is heated for a sufficient time at the desired temperature, recovery of the cannabinoid nanoparticle from the dispersion can include various steps. In some aspects, for example, the cannabinoid nanoparticle can solidify or precipitate from the dispersion as the volume of the dispersion reduces to a certain level during heating. In one aspect, the dispersion can be heated at a temperature equivalent to from about 210° F. to about 280° F. at a pressure of 1 atm (e.g., equivalent to from about 220° F. to about 240° F. at 1 atm, or equivalent to from about 225° F. to about 230° F. at 1 atm) for a time sufficient to reduce the volume of the dispersion by about 10-95%, e.g., about 10%, 20%, 30%, 40%, 50%, 60%, 70%, 75%, 80%, 85%, 90%, or 95%, to thereby solidify or precipitate the cannabinoid nanoparticle from the dispersion.

Optionally, as the volume of the dispersion comprising the sugar, sugar alcohol, or sugar substitute, water, and cannabinoid decreases during the heating step, various additional steps can be performed to aid in the recovery of the cannabinoid nanoparticle from the dispersion. In one aspect, for example, when the volume of the dispersion decreases to the desired level during the heating step, the dispersion can be agitated or stirred while maintaining the dispersion at the desired temperature or temperature range. In a further aspect, when the volume of the dispersion decreases to the desired level during the heating step, the dispersion can be agitated or stirred but not so vigorously as to create a vortex in the dispersion, while maintaining the dispersion at the desired temperature or temperature range. In one aspect, for example, the dispersion comprising the sugar, sugar alcohol, or sugar substitute, water, and cannabinoid can be agitated or stirred when the volume of the dispersion has decreased to the desired level, e.g., decreased by about 10-95%, e.g., about 10%, 20%, 30%, 40%, 50%, 60%, 70%, 75%, 80%, 85%, 90%, or 95%, to thereby solidify or precipitate the cannabinoid nanoparticle from the dispersion.

In one aspect, as the volume of the dispersion comprising the sugar, sugar alcohol, or sugar substitute, water, and cannabinoid decreases during the heating step, additional sugar, sugar alcohol, or sugar substitute can be added to the dispersion to aid in the solidification (e.g., by, optionally seeding the formation of a precipitate) of the cannabinoid nanoparticle. For example, according to one aspect, additional sugar, sugar alcohol, or sugar substitute can be added in increments, e.g., increments of about 0.2 to about 0.5 grams, along with agitation or stirring, until visible solidification (e.g., precipitation) of the cannabinoid nanoparticle from the dispersion is observed. In a further aspect, solidification of the cannabinoid nanoparticle from the dispersion can be aided by the use of an additive such as an antisolvent, e.g., a solvent that will not readily dissolve the cannabinoid nanoparticle.

In some aspects, once solidification of the cannabinoid nanoparticle from the dispersion has been observed, the dispersion can remain at the desired temperature equivalent to from about 210° F. to about 280° F. at 1 atm (e.g., equivalent to from about 220° F. to about 240° F. at 1 atm, or equivalent to from about 225° F. to about 230° F. at 1 atm) for a time sufficient to evaporate most or all of the liquid remaining in the dispersion. In another aspect, once solidification has been observed, the dispersion can be removed from the heat source with continuous agitation or stirring until the liquid remaining in the dispersion evaporates and the dispersion slowly cools. According to one aspect, for example, when the dispersion begins to attain the consistency of a slurry, the dispersion can be removed from the heat source and continuously agitated or stirred until the slurry resembles wet sand. Additional stirring or agitation can be performed until the slurry comprising the cannabinoid nanoparticle dries to a solid, granular form. Alternatively, according to other aspects, once solidification of the cannabinoid nanoparticle occurs, e.g., when the dispersion begins to attain the consistency of a slurry, the dispersion can be filtered through a suitable filter to provide a filtride comprising the cannabinoid nanoparticle. Similarly, in some aspects, once solidification of the cannabinoid nanoparticle has occurred, the dispersion can be dried according to methods known in the art, e.g., drying under reduced pressure.

In a further aspect, the dispersion comprising the cannabinoid nanoparticle can be cooled after the heating step (i.e., in which the dispersion is heated at a temperature equivalent to from about 210° F. to about 280° F. at 1 atm). In one aspect, for example, the dispersion can be heated at the desired temperature until there is no visible liquid (i.e., until the dispersion attains the consistency of a slurry), or until solid nanoparticle formation begins to occur. The dispersion can then be removed from the heat and placed on a cooling tray with a larger surface area to facilitate uniform cooling (e.g., a stainless steel surface). This can in some aspects reduce the potential for the solid nanoparticle to adhere to the heating vessel, which can result in lower yields. The cooling surface comprising the dispersion can be kept at a temperature generally less than 180° F. until the product appears dry and granular. In some aspects, the cooling surface can then be kept at a temperature of about 160° F. or below for a few minutes or a sufficient time to enable to nanoparticle product to solidify and cool.

Optionally, depending on the desired application, the dried cannabinoid nanoparticle obtained from the dispersion can be ground into a powder. A powder of the cannabinoid nanoparticle can be formed using methods known in the art, such as for example through the use of a food processor. In some aspects, once the cannabinoid nanoparticle has been recovered from the dispersion, the solid cannabinoid nanoparticle can be stored in a cool, dark, and dry environment until further use. In one aspect, for example, the cannabinoid nanoparticle can be stored at a temperature equivalent to about 75° F. or less at a pressure of 1 atm (i.e., room temperature or below).

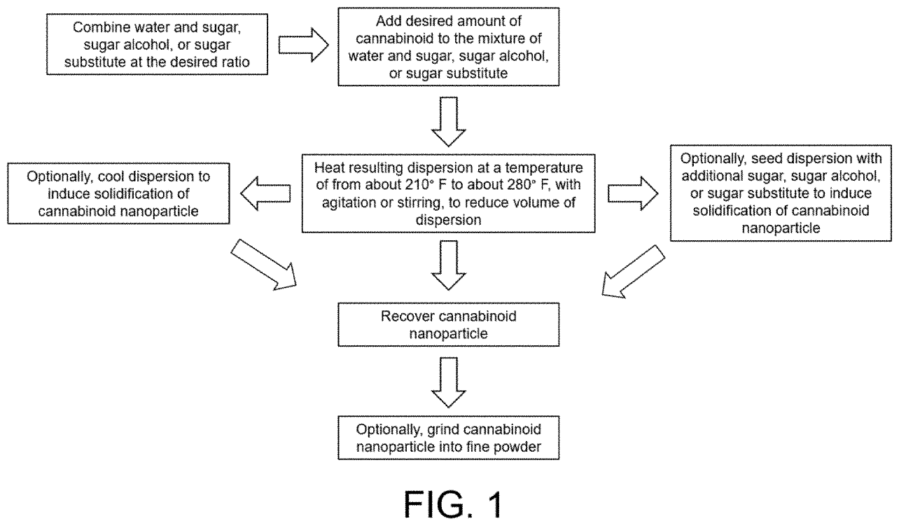

Referring now to FIG. 1, in one aspect, the method for making the cannabinoid nanoparticle can comprise combining water and the sugar, sugar alcohol, or sugar substitute at the desired ratio, followed by adding the desired amount of cannabinoid to the mixture of water and the sugar, sugar alcohol, or sugar substitute. The resulting dispersion can be heated at a temperature equivalent to from about 210° F. to about 280° F. at a pressure of 1 atm for a sufficient time to reduce the volume of the dispersion. As the volume of the dispersion decreases, the cannabinoid nanoparticle can solidify from the dispersion, depending on the dispersion composition. If the cannabinoid nanoparticle does not readily solidify, optional steps can be carried out in which the dispersion is optionally seeded with additional sugar, sugar alcohol, or sugar substitute. Additionally, in some aspects, the dispersion can be cooled to induce solidification of the cannabinoid nanoparticle. Once solidification of the cannabinoid nanoparticle has occurred, the nanoparticle can be recovered from the dispersion and optionally ground into a fine powder suitable for use in a consumer product such as those described below.

3. Composition of the Dispersion

In one aspect, the cannabinoid nanoparticles can be water- and/or alcohol-soluble and thus suitable for infusion into a water-based or alcohol-based comestible. Surprisingly, the inventors discovered that formation of the water- and/or alcohol-soluble cannabinoid nanoparticle can be obtained from a dispersion that is substantially free of any solvent other than water, e.g., a dispersion comprising less than about 10% by weight of any non-water-based solvent such as an organic solvent (e.g., hexane, alcohols, and the like). For example, according to one aspect, the dispersion can be substantially free of alcohol, e.g., comprise less than about 10% by weight of alcohol, relative to the total weight of the dispersion. Thus, in some aspects, the cannabinoid nanoparticle can be recovered from the dispersion without the use of an alcohol such as isopropyl alcohol or ethanol.

In a further aspect, the dispersion comprising the sugar, sugar alcohol, or sugar substitute, water, and cannabinoid can be substantially free of ethanol. In one aspect, the dispersion can comprise less than about 10%, less than about 5%, less than about 1%, less than about 0.5%, less than about 0.1%, less than about 0.05%, or less than about 0.01% of ethanol.

In other aspects, the dispersion comprising the sugar, sugar alcohol, or sugar substitute, water, and cannabinoid can be substantially free of isopropyl alcohol. In one aspect, for example, the dispersion can comprise less than about 10%, less than about 5%, less than about 1%, less than about 0.5%, less than about 0.1%, less than about 0.05%, or less than about 0.01% of isopropyl alcohol.

In a further aspect, the dispersion comprising the sugar, sugar alcohol, or sugar substitute, water, and cannabinoid can be substantially free of ethanol and isopropyl alcohol, e.g., comprise less than about 10%, less than about 5%, less than about 1%, less than about 0.5%, less than about 0.1%, less than about 0.05%, or less than about 0.01% of ethanol and isopropyl alcohol.

In another aspect, the dispersion can be free of ethanol. In a further aspect, the dispersion can be free of isopropyl alcohol. In a still further aspect, the dispersion can be free of ethanol and isopropyl alcohol. Similarly, in some aspects, the dispersion can be free of any organic solvent, including for example hexane or alcohol-based solvents.

In one aspect, the cannabinoid nanoparticle can be recovered from the dispersion comprising the sugar, sugar alcohol, or sugar substitute, water, and cannabinoid without the use of a carrier oil to improve the solubility of the cannabinoid, e.g., without creating an emulsion or other multi-phase system in the dispersion. Thus, in some aspects, the dispersion comprising the sugar, sugar alcohol, or sugar substitute, water, and cannabinoid is free of any carrier oil, including but not limited to medium chain triglyceride (MCT) oil, long chain triglyceride (LCT) oil, vegetable oil, canola oil, olive oil, sunflower oil, coconut oil (including fractionated coconut oil), hemp oil, palm oils, and/or other oils suitable for human or animal consumption. Similarly, the dispersion comprising the sugar, sugar alcohol, or sugar substitute, water, and cannabinoid can be free of other water-soluble agents used for creating an emulsion in the dispersion or improving the solubility of the cannabinoid, including without limitation a starch such as a modified food starch, gum arabic, quillaja extract, or cyclodextrin. In some aspects, the dispersion can be similarly free of a sugar alcohol or maltodextrin, as further described below.

In a further aspect, the dispersion from which the cannabinoid nanoparticle can be recovered consists essentially of the sugar, sugar alcohol, or sugar substitute, the water, and the cannabinoid. In one aspect, as discussed below, the dispersion is free of a sugar alcohol, and thus the dispersion can consist essentially of the sugar or sugar substitute, the water, and the cannabinoid. Similarly, in some aspects, the dispersion is free of a sugar alcohol and sugar substitute and consists essentially of the sugar, the water, and the cannabinoid.

In a still further aspect, the dispersion from which the cannabinoid nanoparticle can be recovered consists of the sugar, sugar alcohol, or sugar substitute, the water, and the cannabinoid. In one aspect, as discussed below, the dispersion is free of a sugar alcohol, and thus the dispersion can consist of the sugar or sugar substitute, the water, and the cannabinoid. Similarly, in some aspects, the dispersion is free of a sugar alcohol and sugar substitute and consists of the sugar, the water, and the cannabinoid.

a. Sugars, Sugar Alcohols, and Sugar Substitutes

A variety of sugars, sugar alcohols, and sugar substitutes are contemplated for use in the dispersion. In one aspect, the sugar, when present in the dispersion, can comprise allulose, glucose, dextrose, fructose, galactose, sucrose, lactose, maltose, trehalose, maltodextrin, or a combination thereof. In a further aspect, the sugar, when present in the dispersion, can comprise sucrose, fructose, glucose, or a combination thereof. In a still further aspect, the sugar, when present in the dispersion, can comprise sucrose, fructose, and glucose. In one aspect, for example, the sugar can be a naturally-occurring sugar, such as cane sugar, which comprises sucrose, fructose, and glucose.

In one aspect, sugar alcohols suitable for use with the dispersion can include without limitation ethylene glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, polyglycitol, or a combination thereof. In a further aspect, the sugar alcohol, when present in the dispersion, can comprise erythritol, xylitol, or a combination thereof.

In another aspect, the dispersion is substantially free of a sugar alcohol. According to one aspect, for example, the dispersion can comprise less than about 10%, less than about 5%, less than about 1%, less than about 0.5%, less than about 0.1%, less than about 0.05%, or less than about 0.01% of any sugar alcohol. In one aspect, the dispersion can be substantially free of glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, and polyglycitol. In a further aspect, the dispersion can be substantially free of isomalt, mannitol, sorbitol, xylitol, lactitol, maltitol, and erythritol.

In one aspect, the dispersion is free of a sugar alcohol. Thus, for example, according to one aspect, the dispersion is free of glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, and polyglycitol. In a further aspect, the dispersion is free of isomalt, mannitol, sorbitol, xylitol, lactitol, maltitol, and erythritol.

A variety of sugar substitutes are also contemplated for use with the dispersion. Suitable plant-derived sugar substitutes include without limitation brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, xylitol (also known as a sugar alcohol), or a combination thereof.

Suitable artificial sugar substitutes contemplated for use with the dispersion include without limitation acesulfame potassium, advantame, alitame, aspartame, salts of aspartame-acesulfame, sodium cylclamate, dulcin, glucin, neohesperidin dihidryochalcone, neotame, P-4,000, saccharin, sucralose, or a combination thereof. In a further aspect, the dispersion can comprise an artificial sweetener comprising sucralose.

In another aspect, the dispersion is substantially free of any sugar substitute. According to one aspect, for example, the dispersion can comprise less than about 10%, less than about 5%, less than about 1%, less than about 0.5%, less than about 0.1%, less than about 0.05%, or less than about 0.01% of any sugar substitute. In one aspect, the dispersion can be substantially free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be substantially free of acesulfame potassium, advantame, alitame, aspartame, salts of aspartame-acesulfame, sodium cylclamate, dulcin, glucin, neohesperidin dihidryochalcone, neotame, P-4,000, saccharin, and sucrolose.

In another aspect, the dispersion is free of any sugar substitute. In one aspect, for example, the dispersion can be free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be free of acesulfame potassium, advantame, alitame, aspartame, salts of aspartame-acesulfame, sodium cylclamate, dulcin, glucin, neohesperidin dihidryochalcone, neotame, P-4,000, saccharin, and sucrolose.

b. Cannabinoids

Cannabis is a genus of flowering plants that includes at least three species, Cannabis sativa, Cannabis indica, and Cannabis ruderalis. Cannabis plants produce a family of terpeno-phenolic compounds called cannabinoids. More than 100 cannabinoids have been identified from crude cannabis. Most cannabinoids exist in two forms, as acids and in neutral (decarboxylated) forms. The acid form is designated by an “A” at the end of its acronym, e.g., TCHA. Cannabinoids are synthesized in the plant as acid forms, and while some decarboxylation does occur in the plant, it increases significantly post-harvest, and the kinetics of decarboxylation increase at high temperatures. Decarboxylation can be achieved by thorough drying of the plant material followed by heating it or exposing it to light or alkaline conditions.

In various aspects, the disclosed dispersions include one or more cannabinoids. The cannabinoids can be in the acid or neutral form and can be derived from a cannabis plant or produced synthetically. Thus, in various aspects, the cannabinoid can be a cannabinoid acid. In some aspects, the cannabinoid in the dispersion can be Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), Δ⁹-tetrahydrocannabiphorol (Δ⁹-THCP), cannabichromene (CBC), cannabicyclol (CBL), cannabidiol (CBD), cannabidiphorol (CBDP), cannabielsoin (CBE), cannabigerol (CBG), cannabinidiol (CBND), cannabinol (CBN), cannabitriol (CBT), or a combination thereof. The cannabinoid in the dispersion can also be in acid form, e.g., Δ⁹-THCA, Δ⁸-THCA, CBCA, CBLA, CBDA, CBEA, CBGA, CBNDA, CBNA, CBTA, or a combination thereof. In some aspects, the cannabinoid is CBD, CDPB, CBDA, THC, THCP, THCA, or a combination thereof. In further aspects, the cannabinoid is CBD, THC, or a combination thereof. In further aspects, the cannabinoid is CBD. In still further aspects, the cannabinoid is THC.

In other aspects, the dispersion is substantially free of THC (including Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), and Δ⁹-tetrahydrocannabiphorol (Δ⁹-THCP)). In some aspects, the dispersion has less than about 10 wt % THC, less than about 5 wt % THC, less than about 3 wt % THC, less than about 1 wt % THC, less than about 0.8 wt % THC, less than about 0.7 wt % THC, less than about 0.6 wt % THC, less than about 0.5 wt % THC, less than about 0.4 wt % THC, less than about 0.3 wt % THC, less than about 0.2 wt % THC, less than about 0.1 wt % THC, less than about 0.05 wt % THC, or less than about 0.01 wt % THC, based on the total weight of the dispersion. In a further aspect, the dispersion is free of THC.

In some aspects, the dispersion is substantially free of CBN. In some aspects, the dispersion has less than about 10 wt % CBN, less than about 5 wt % CBN, less than about 3 wt % CBN, less than about 1 wt % CBN, less than about 0.8 wt % CBN, less than about 0.7 wt % CBN, less than about 0.6 wt % CBN, less than about 0.5 wt % CBN, less than about 0.4 wt % CBN, less than about 0.3 wt % CBN, less than about 0.2 wt % CBN, less than about 0.1 wt % CBN, less than about 0.05 wt % CBN, or less than about 0.01 wt % CBN, based on the total weight of the dispersion. In a further aspect, the dispersion is free of CBN.

In further aspects, the dispersion is substantially free of THC and CBN. Thus, in various further aspects, the dispersion has less than about 10 wt %, less than about 5 wt %, less than about 3 wt %, less than about 1 wt %, less than about 0.8 wt %, less than about 0.7 wt %, less than about 0.6 wt %, less than about 0.5 wt %, less than about 0.4 wt %, less than about 0.3 wt %, less than about 0.2 wt %, less than about 0.1 wt %, less than about 0.05 wt %, or less than about 0.01 wt %, of both THC and CBN, based on the total weight of the dispersion. In a further aspect, the dispersion is free of THC and CBN.

In some aspects, the cannabinoid in the dispersion can have a purity of at least 90%. In a further aspect, the purity of the cannabinoid in the dispersion ranges from 90-100%. In a still further aspect, the purity of the cannabinoid in the dispersion ranges from 92-97%. In general, the inventors have discovered that the encapsulation process described herein can be improved with the use of a pure cannabinoid starting material (e.g., from 92-97% pure). Similarly, the nanoparticle encapsulated cannabinoid products can advantageously maintain the purity of the cannabinoid over extended periods of time. In some aspects, the purity of the cannabinoid starting material can be maintained once encapsulated for a period of twelve months or longer, e.g., 18 months or longer, or even 24 months or longer. The stable shelf life of the cannabinoid nanoparticles is an advantage over many existing methods for delivering cannabinoid products. In some aspects, the stability of the cannabinoid can be measured using NMR, e.g., by comparing a pre-encapsulated cannabinoid to a cannabinoid extracted from a nanoparticle prepared by a disclosed method.

In various aspects, the one or more cannabinoids in the dispersion can be obtained commercially, prepared synthetically, or extracted from a cannabis plant. In other aspects, synthetic biology methods can be used to prepare the cannabinoids, e.g., through microbial factories. Once harvested, cannabis plant material typically includes flowers, leaves, and/or stems. In some aspects, cannabis plant material can be frozen for a suitable period of time, e.g., 36 hours, prior to being dried and extracted. Once dried, cannabis plant material can be extracted using a variety of techniques, including hydrocarbon extraction and supercritical CO₂ extraction.

In some aspects, the dispersion can comprise neutral cannabinoids, which can be prepared by decarboxylating cannabinoid acids. In various aspects, cannabinoid acids obtained from cannabis plant material can be decarboxylated by heating the dried plant material at a temperature of about 220° F. for at least 10-15 minutes followed by heating for about 280° F. for at least 45 minutes. Other known methods for decarboxylating cannabinoid acids from cannabis plant material can also be used.

According to one aspect, the cannabinoid in the dispersion can be provided from a cannabinoid composition that comprises a certain amount of cannabinoid. In some aspects, for example, the cannabinoid can be provided from a composition that comprises at least about 90% by weight of a cannabinoid. In a further aspect, the cannabinoid can be provided from a composition that comprises at least about 95% by weight of a cannabinoid. In a still further aspect, the cannabinoid can be provided from a composition that comprises at least about 99% by weight of a cannabinoid. For example, in one aspect, the cannabinoid is provided from commercially-available CBD isolate, which is typically a crystalline or solid powder comprising 99% CBD by weight.

In a further aspect, one or more cannabinoids can be in the dispersion and final cannabinoid nanoparticle. Thus, for example, the dispersion and final cannabinoid nanoparticle can comprise two or more, three or more, or four or more cannabinoids. In one aspect, the dispersion and final cannabinoid nanoparticle can comprise two different cannabinoids

c. Other Additives

In some aspects, the disclosed dispersion can comprise additional ingredients that can be encapsulated with the nanoparticle, along with the cannabinoid. In one aspect, the dispersion comprises a nutraceutical. Any suitable nutraceutical additive can be present in the dispersion. In some aspects, the nutraceutical additive can be Vitamin E, Echinacea, St. John's Wart, Kava Tincture, Ginseng, Black Seed Oil, Milk Thistle, Turmeric, Blank-Nega, Valerian root extract, among others. In a further aspect, the dispersion can comprise a suitable flavor additive such as a natural or artificial flavoring.

In some aspects, the disclosed compositions and nanoparticles can comprise further additives. Such additives can be added during the encapsulation process, in which the further additive(s) can be potentially encapsulated, or such additives can be mixed with the final encapsulated product. Alternatively, one or more further additives can be encapsulated using a disclosed process and then mixed with cannabinoid-encapsulated nanoparticles. Such further additives include Ginger, Lemongrass, Yucca extract, Orris root, Black Tea, Green Tea, Other tea extracts or powders like Matcha powder and the like, natural essential oils and oil extracts used in the food and/or pharmaceutical industry, Food-grade nut extracts, Olive oil such as Extra virgin olive oil, Coconut oil, Lime and/or lemon extract, Fruit and berry extracts and fruit/berry dry powders, Flower extracts and Flower dry powders, Cinnamon, Cumin, and other spices, Soya Lecithin and other food-grade surfactants/emulsifiers, Guar Gum, β-glucans and other sugars, Malt extract, Mowrah butter, Kelp powder, Astaxanthin and other carotenoids, Natural Food supplements and flavorings, Coffee extract, Cocoa extract, water-soluble vitamins (Thiamine, Riboflavin, Pantothenicc acid, Niacin, Pyridoxine, Folcin or Folic acid, Cyanocobalamin, Ascorbic Acid, among others), and multivitamin mixtures.

d. Acceptable Salts and Carriers

In some aspects, a naturally-occurring cannabinoid or sugar present in the dispersion or a product prepared therefrom can be present as an acceptable, non-naturally occurring salt. Thus, a naturally occurring cannabinoid or sugar present in the composition can be present as a non-naturally occurring acid or base salt of the naturally occurring cannabinoid or sugar. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

Acceptable salts can be prepared by reaction of the cannabinoid or sugar with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates.

According to one aspect, if the cannabinoid or sugar has one or more acidic functional groups, the desired salt can be prepared by any suitable method known in the art, for example, treatment of the free acid with an inorganic or organic base, such as an amine (primary, secondary or tertiary), an alkali metal hydroxide or alkaline earth metal hydroxide, or the like. It is understood that the acceptable salts are non-toxic and suitable for ingestion. Additional information on suitable acceptable salts can be found in Remington's Pharmaceutical Sciences, 17th ed., Mack Publishing Company, Easton, Pa., 1985, which is incorporated herein by reference.

In some aspects, a product prepared from a disclosed dispersion can comprise a naturally-occurring cannabinoid and/or sugar present along with an acceptable, non-naturally occurring carrier. Various suitable non-naturally occurring carriers are described in Remington's Pharmaceutical Sciences, 17th ed., Mack Publishing Company, Easton, Pa., 1985, which is incorporated herein by reference. Non-limiting examples include non-naturally occurring polymeric carriers or binders in liquid or solid form, such as polyglycolic acids, synthetic polymers, non-naturally occurring conjugates of proteins, and the like.

e. Exemplary Dispersions

Specific, non-limiting examples of dispersions useful for preparing the cannabinoid nanoparticle include those listed in Table 1. The compositions listed in Table 1 include dispersions comprising a sugar, sugar alcohol, or sugar substitute selected from a sugar comprising sucrose, fructose, and glucose (e.g., cane sugar or organic cane sugar); a sugar alcohol selected from erythritol or xylitol; maltodextrin, or sucralose. In one aspect, the exemplary dispersion compositions listed in Table 1 include a sugar comprising sucrose, fructose, and glucose such as cane sugar or organic cane sugar. In various aspects, the CBD listed in Table 1 can be CBD isolate, e.g., CBD isolate comprising at least about 99% CBD. The cannabinoids listed in Table 1 can be present in the composition alone or in any combination.

TABLE 1
EXEMPLARY DISPERSION COMPOSITIONS

| | Mass Ratio of | Mass Ratio of |
|---|---|---|
| | Cannabinoid to | Sugar/Sugar |
| | Sugar/Sugar | Alcohol/Sugar |
| | Alcohol/Sugar | Substitute to |
| Cannabinoid | Substitute | Water |
| CBD, CBDP, CBG, | 1:300 to 1:5 | 1:8 to 1:1.2 |
| THC, THCP, or CBN | | |
| CBD, CBDP, CBG, | 1:300 to 1:5 | 1:8 to 1:1.3 |
| THC, THCP, or CBN | | |
| CBD, CBDP, CBG, | 1:300 to 1:5 | 1:8 to 1:1.5 |
| THC, THCP, or CBN | | |
| CBD, CBDP, CBG, | 1:300 to 1:5 | 1:6 to 1:4 |
| THC, THCP, or CBN | | |
| CBD, CBDP, CBG, | 1:300 to 1:5 | 1:4 |
| THC, THCP, or CBN | | |
| CBD, CBDP, CBG, | 1:50 to 1:5 | 1:8 to 1:1.2 |
| THC, THCP, or CBN | | |
| CBD, CBDP, CBG, | 1:50 to 1:10 | 1:8 to 1:1.2 |
| THC, THCP, or CBN | | |
| CBD, CBDP, CBG, | 1:40 to 1:10 | 1:8 to 1:1.2 |
| THC, THCP, or CBN | | |
| CBD, CBDP, CBG, | 1:30 to 1:10 | 1:8 to 1:1.2 |
| THC, THCP, or CBN | | |
| CBD, CBDP, CBG, | 1:30 to 1:10 | 1:8 to 1:1.2 |
| THC, THCP, or CBN | | |
| CBD, CBDP, CBG, | 1:30 to 1:15 | 1:8 to 1:1.2 |
| THC, THCP, or CBN | | |
| CBD, CBDP, CBG, | 1:20 | 1:8 to 1:1.2 |
| THC, THCP, or CBN | | |
| CBD, CBDP, CBG, | 1:50 to 1:10 | 1:8 to 1:1.3 |
| THC, THCP, or CBN | | |
| CBD, CBDP, CBG, | 1:50 to 1:10 | 1:8 to 1:1.5 |
| THC, THCP, or CBN | | |
| CBD, CBDP, CBG, | 1:50 to 1:10 | 1:6 to 1:4 |
| THC, THCP, or CBN | | |
| CBD, CBDP, CBG, | 1:50 to 1:10 | 1:4 |
| THC, THCP, or CBN | | |
| CBD, CBDP, CBG, | 1:30 to 1:10 | 1:8 to 1:1.3 |
| THC, THCP, or CBN | | |
| CBD, CBDP, CBG, | 1:30 to 1:10 | 1:8 to 1:1.5 |
| THC, THCP, or CBN | | |
| CBD, CBDP, CBG, | 1:30 to 1:10 | 1:6 to 1:4 |
| THC, THCP, or CBN | | |
| CBD, CBDP, CBG, | 1:30 to 1:10 | 1:4 |
| THC, THCP, or CBN | | |
| CBD, CBDP, CBG, | 1:30 to 1:15 | 1:8 to 1:1.3 |
| THC, THCP, or CBN | | |
| CBD, CBDP, CBG, | 1:30 to 1:15 | 1:8 to 1:1.5 |
| THC, THCP, or CBN | | |
| CBD, CBDP, CBG, | 1:30 to 1:15 | 1:6 to 1:4 |
| THC, THCP, or CBN | | |
| CBD, CBDP, CBG, | 1:30 to 1:15 | 1:4 |
| THC, THCP, or CBN | | |
| CBD, CBDP, CBG, | 1:20 | 1:8 to 1:1.3 |
| THC, THCP, or CBN | | |
| CBD, CBDP, CBG, | 1:20 | 1:8 to 1:1.5 |
| THC, THCP, or CBN | | |
| CBD, CBDP, CBG, | 1:20 | 1:6 to 1:4 |
| THC, THCP, or CBN | | |
| CBD, CBDP, CBG, | 1:20 | 1:4 |
| THC, THCP, or CBN | | |

In some aspects, the cannabinoid nanoparticles have a particle size that is much lower than existing cannabinoid delivery systems, enabling better bioabsorption among other beneficial properties. In some aspects, the disclosed nanoparticles have a size ranging from about 200 nm to about 2,000 nm, e.g., from about 200 nm to about 1,000 nm, or from about 200 nm to about 500 nm. Particle size can be determined using methods known in the art, e.g., light scattering or zeta potential measurements. Particle size, as referred to herein, refers to the mean or average particle size of a given cannabinoid nanoparticle sample. The inventors have surprisingly discovered that the disclosed method enables the production of cannabinoid nanoparticles that are 1,000 times smaller than other existing cannabinoid delivery systems.

### C. Products Comprising the Cannabinoid Nanoparticles

Also described herein are products comprising the cannabinoid nanoparticle prepared by a disclosed method. One advantage of the disclosed method is that it allows for the infusion of a cannabinoid into a comestible product that masks the taste of the cannabinoid. In one aspect, for example, a water-based or alcohol-based comestible can comprise the cannabinoid nanoparticle prepared by a disclosed method, together with a water-based or alcohol-based liquid. In another aspect, the cannabinoid nanoparticle can be at least partially dissolved in the comestible liquid, e.g., water-based or alcohol-based liquid. In a further aspect, the comestible liquid such as a water-based or alcohol-based liquid can comprise the cannabinoid nanoparticle uniformly dispersed therein. In a further aspect, a solid or semi-solid comestible can comprise the cannabinoid nanoparticle prepared by a disclosed method, together with one or more foodstuff ingredients.

In various aspects, the cannabinoid nanoparticle prepared by a disclosed method can be infused into a water-based or alcohol-based comestible by mixing the cannabinoid nanoparticle into the liquid, e.g., by stirring or agitating the mixture until no solid cannabinoid nanoparticle particles are observed. The cannabinoid nanoparticle can be infused into a variety of beverages, including without limitation flavored or unflavored carbonated or still water, beer, wine, hard seltzer, cocktails, and the like.

1. Solid and Semi-Solid Comestibles

In further aspects, the cannabinoid nanoparticle prepared by a disclosed method can be infused into a solid or semi-solid comestible. Examples of suitable comestibles include without limitation vitamins, supplements, concentrates, extracts, capsules, tablets, powders, lozenges, chewing gums, chewable candies, hard candies, cakes, chocolate bars, granola bars, nut bars, and the like. The comestible food product can comprise one or more foodstuff ingredients, including without limitation starch, sugar, sugar alcohols, nuts, eggs, milk, chocolate powder, cream, water, emulsifiers, food preservatives, and other ingredients common in food. In a further aspect, the cannabinoid nanoparticles prepared by a disclosed method can be incorporated into various comestible sex aids, including without limitation creams, gels, oils, and the like.

In other aspects, the cannabinoid nanoparticle prepared by a disclosed method can be incorporated into a comestible product suitable for animal or pet, such as a dog. Examples include without limitation flavored pet or dog treats, chews, bones, biscuits, cookies, jerkies, and the like. Without wishing to be bound by theory, it is believed that the cannabinoid nanoparticles prepared by a disclosed method can be useful for helping with an animal or pet's anxiety, stress, pain, inflammation, arthritis, seizures, digestion problems, among others.

2. Products for Therapeutic Uses

In further aspects, the cannabinoid nanoparticle prepared by a disclosed method can be incorporated into a comestible product for therapeutic use, including products suitable for humans and animals. Without wishing to be bound by theory, the cannabinoid nanoparticles can be useful for treating a variety of conditions, disorders, and illnesses. Examples include without limitation lack of appetite, stress, anxiety, depression, nausea, motion sickness, vomiting, weight loss, inflammation, arthritis, gout, epilepsy, addiction, drug or alcohol dependence, pain, attention deficit disorder (ADD), autism/Asperger's disorder, psychiatric disorders including bipolar disorder, mania, obsessive compulsive disorder (OCD), insomnia, digestive disorders, among others.

In various aspects, the cannabinoid nanoparticle prepared by a disclosed method can be formulated as a composition or formulation comprising a suitable carrier. Non-limiting examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and organic esters such as ethyl oleate. Proper fluidity can be maintained, for example, by the use of coating materials such as lecithin, by the maintenance of the required particle size in the case of dispersions and by the use of surfactants.

Suitable carriers for a product for therapeutic use can also comprise adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Suitable inert carriers can include sugars such as lactose.

In some aspects, the product for a therapeutic use can include an excipient. Suitable excipients include, without limitation, saccharides, for example, glucose, lactose, or sucrose, mannitol, or sorbitol, cellulose derivatives, and/or calcium phosphate, for example, tricalcium phosphate or acidic calcium phosphate.

In further aspects, the product for a therapeutic use can include a binder. Suitable binders include, without limitation, tare compounds such as starch paste, for example, corn, wheat, rice, and potato starch, gelatin, tragacanth, methylcellulose, hydroxypropyl methylcellulose, carboxymethylcellulose, and/or polyvinylpyrrolidone. In still further aspects, there can be a disintegrating agent, such as the aforementioned starches and carboxymethyl starch, crosslinked polyvinylpyrrolidone, agar, or alginic acid or a salt thereof, such as sodium alginate.

In some aspects, the product for a therapeutic use can include an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, or silica. In a further aspect, the additive can include flowability-control agents and lubricants, such as silicon dioxide, talc, stearic acid and salts thereof, such as magnesium stearate or calcium stearate, and/or propylene glycol.

The therapeutic product can be formulated for oral use, such as for example, a tablet, pill, or capsule, and the composition can include a coating layer that is resistant to gastric acid. Such a layer, in various aspects, can include a concentrated solution of saccharides that can comprise gum arabic, talc, polyvinylpyrrolidone, polyethylene glycol, and/or titanium dioxide, and suitable organic solvents or salts thereof.

The effective amount of the cannabinoid in the therapeutic product can vary within wide limits. Such a dosage can be adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted in the event of any contraindications. Dosage can vary, and can be administered in one or more dose administrations daily, for one or several days.

### D. Methods for Treating a Disorder

In one aspect, disclosed is a method of treating a condition in a subject, comprising administering to the subject the cannabinoid nanoparticle prepared by a disclosed method. In a further aspect, disclosed is a method of treating a condition in a subject, comprising administering to the subject the product of a disclosed method, a disclosed water-based or alcohol-based comestible, or a disclosed solid or semi-solid comestible.

According to one aspect, the subject is a mammal. In a further aspect, the subject is a human. In a still further aspect, the subject has been diagnosed with a need for treatment of the disorder prior to the administering step. In a further aspect, the subject is at risk for developing the disorder prior to the administering step. In a further aspect, the method further comprises the step of identifying a subject in need of treatment of the disorder.

In various aspects, the disorder is lack of appetite, stress, anxiety, depression, nausea, motion sickness, vomiting, weight loss, inflammation, arthritis, gout, epilepsy, addiction, drug or alcohol dependence, pain, attention deficit disorder (ADD), autism/Asperger's disorder, a psychiatric disorder, bipolar disorder, mania, obsessive compulsive disorder (OCD), insomnia, or a digestive disorder.

The product administered to the subject can be formulated for oral use, such as for example, a tablet, pill, or capsule, and the composition can include a coating layer that is resistant to gastric acid, as described above. Such a layer, in various aspects, can include a concentrated solution of saccharides that can comprise gum arabic, talc, polyvinylpyrrolidone (PVP), polyethylene glycol, and/or titanium dioxide, and suitable organic solvents or salts thereof. In a further aspect, the product can be administered to the subject as a nasal spray, inhaler, tincture, topically applied as a cream, or via syringe or dropper.

The effective amount of the cannabinoid in the product administered to the subject can vary within wide limits. Such a dosage can be adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted in the event of any contraindications. Dosage can vary, and can be administered in one or more dose administrations daily, for one or several days.

### E. Examples

The following examples are put forth so as to provide those of ordinary skill in the art with a complete disclosure and description of how the methods and products claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° F. or is at ambient temperature, and pressure is at or near atmospheric. The Examples are provided herein to illustrate the invention, and should not be construed as limiting the invention in any way.

To prepare the exemplary dispersions, an induction cooktop was used to maintain consistent heat, along with a stainless steel pot as a vessel for the dispersion, and stainless steel or glass utensils for stirring or agitation. The desired amount organic cane sugar and filtered or spring water was first mixed before the addition of the cannabinoid. A desired amount of cannabinoid (e.g., CBD) was then added to the mixture, and the induction cooktop was set at a temperature range of from 212° F. to 280° F. It was observed that setting the temperature of the induction cooktop to a temperature above 280° F. can affect the quality of the cannabinoid nanoparticle recovered from the dispersion. The dispersion was then brought to a boil. After about ¾ of the volume of the original dispersion boiled away, the dispersion was agitated with stirring while keeping the dispersion heated on the induction cooktop. The dispersion was stirred and agitated but not so vigorously as to create a vortex in the dispersion.

Optionally, depending on the ratio of organic cane sugar to the water, an optional seeding step was performed. Incrementally, a pinch (about 0.36 grams) of organic can sugar was added to the dispersion with stirring until visible solidification (e.g., precipitation) of the cannabinoid nanoparticle was observed. The sides of the stainless steel vessel were also scraped to dislodge solidified cannabinoid nanoparticle and to aid in further solidification of the nanoparticle from the dispersion.

The stainless steel vessel comprising the cannabinoid nanoparticle and remaining liquid was then removed from the induction cooktop when most of the liquid had evaporated from the dispersion and the dispersion resembled wet sand. The dispersion was continuously stirred until the remaining liquid evaporated, which yielded granulated solid cannabinoid nanoparticle particles resembling dry sand.

The cannabinoid nanoparticle recovered from the dispersion was then ground into a fine powder using a food processor. The ground nanoparticle was stored in a dry, dark and cool location (less than about 75° F.) until further use.

Using a stir bar or other similar device, the cannabinoid nanoparticle was infused into the desired food or beverage comestible. When a tabletop stir bar or other similar device is used, low to medium speed was sufficient to ensure that all cannabinoid nanoparticle particles were infused into the food or beverage. Infusion was complete when no solid cannabinoid nanoparticle particles were observed in the food or beverage.

Table 2 lists exemplary cannabinoid nanoparticles recovered from dispersions prepared according to the preceding exemplary method.

TABLE 2
EXAMPLE DISPERSIONS/CANNABINOID NANOPARTICLES

| | | | | Mass Ratio of |
|---|---|---|---|---|
| | | | Mass Ratio of | Sugar/Sugar |
| | | | Cannabinoid to | Alcohol/ |
| | | | Sugar/Sugar | Sugar |
| Sugar/Sugar Alcohol/Sugar | Cannabinoid | Water | Alcohol/Sugar | Substitute to |
| Substitute (g) | (g) | (mL) | Substitute | Water |
| Organic Cane Sugar (222.73) | 2-12 (CBD) | 901.01 | 1:111.3 to 1:18.6 | about 1:4 |
| Organic Cane Sugar (55.19) | 0.5 (CBD) | 225.38 | 1:110.4 | about 1:4 |
| Erythritol (55.19)* | 0.5 (CBD) | 225.3 | 1:110.4 | about 1:4 |
| Truvia (Erythritol, Sugar, | 1.0 (CBD) | 225.3 | 1:55.2 | about 1:4 |
| Stevia Leaf Extract) (55.19) | | | | |
| Organic Stevia Extract (35.07) | 0.5 (CBD) | 142.9 | 1:70.1 | about 1:4 |
| Liquid Organic Sugar-Free | 0.5 (CBD) | 225.3 | 1:110.4 | about 1:4 |
| Stevia (Deionized Water, | | | | |
| Organic Stevol Glycosides | | | | |
| from Stevia Leaf, 11% Organic | | | | |
| Alcohol) (55.19) | | | | |
| Swerve (Erythritol, | 1.0 (CBD) | 225.3 | 1:55.2 | about 1:4 |
| Oligosaccharides, Natural | | | | |
| Flavors) (55.19) | | | | |
| Sucralose/Maltodextrin* | 0.5 (CBD) | 225.3 | 1:110.4 | about 1:4 |
| (55.19) | | | | |
| Sucralose/Maltodextrin (55.19) | 1.0 (CBD) | 225.3 | 1:55.2 | about 1:4 |
| Monk Fruit Sweetener with | 0.5 (CBD) | 225.3 | 1:110.4 | about 1:4 |
| Erythritol (55.19) | | | | |
| Superlose (Granular Allulose, | 1.0 (CBD) | 225.3 | 1:55.2 | about 1:4 |
| Monk Fruit Extract, Stevia | | | | |
| Extract, Natural Flavors) | | | | |
| (55.19) | | | | |
| Organic Cane Sugar (75.19) | 10 (Δ⁸-THC) | 225.3 | 1:7.5 | about 1:4 |

*dispersion was cooled and seeded before solidification of cannabinoid nanoparticle was observed

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for making a cannabinoid nanoparticle, the method comprising recovering the nanoparticle from a dispersion comprising:
a) a sugar, sugar alcohol, or sugar substitute;
b) water; and
c) a cannabinoid;
wherein the mass ratio of the cannabinoid to the sugar, sugar alcohol, or sugar substitute is from about 1:300 to about 1:5, and the dispersion comprises less than 10% by weight of alcohol.

2. The method of claim 1, wherein recovering the nanoparticle comprises heating the dispersion at a temperature equivalent to from about 210° F. to about 280° F. at a pressure of 1 atm for a sufficient time to reduce the volume of the dispersion.

3. The method of claim 2, further comprising cooling the dispersion after the heating step.

4. The method of claim 1, wherein the mass ratio of the cannabinoid to the sugar, sugar alcohol, or sugar substitute is from about 1:50 to 1:10.

5. The method of claim 1, wherein the mass ratio of the cannabinoid to the sugar, sugar alcohol, or sugar substitute is about 1:20.

6. The method of claim 1, wherein the mass ratio of the sugar, sugar alcohol, or sugar substitute to the water is from about 1:8 to about 1:1.5 prior to the recovering step.

7. The method of claim 1, wherein the mass ratio of the sugar, sugar alcohol, or sugar substitute to the water is from about 1:6 to about 1:4 prior to the recovering step.

8. The method of claim 1, wherein the mass ratio of the sugar, sugar alcohol, or sugar substitute to the water is about 1:4 prior to the recovering step.

9. The method of claim 1, further comprising forming a powder of the nanoparticle after the recovering step.

10. The method of claim 1, wherein the dispersion is substantially free of ethanol.

11. The method of claim 1, wherein the dispersion is substantially free of isopropyl alcohol.

12. The method of claim 1, wherein the dispersion is free of ethanol.

13. The method of claim 1, wherein the dispersion is free of isopropyl alcohol.

14. The method of claim 1, wherein the dispersion is free of a carrier oil.

15. The method of claim 1, wherein the dispersion consists essentially of the sugar, sugar alcohol, or sugar substitute, the water, and the cannabinoid.

16. The method of claim 1, wherein the dispersion consists of the sugar, sugar alcohol, or sugar substitute, the water, and the cannabinoid.

17. The method of claim 1, wherein the sugar, when present, comprises allulose, glucose, dextrose, fructose, galactose, sucrose, lactose, maltose, trehalose, maltodextrin, or a combination thereof.

18. The method of claim 1, wherein the cannabinoid is provided from a composition that comprises at least about 90% by weight of a cannabinoid.

19. The method of claim 1, wherein the cannabinoid is selected from Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), Δ⁹-tetrahydrocannabiphorol (Δ⁹-THCP), cannabichromene (CBC), cannabicyclol (CBL), cannabidiol (CBD), cannabidiphorol (CBDP), cannabielsoin (CBE), cannabigerol (CBG), cannabinidiol (CBND), cannabinol (CBN), cannabitriol (CBT), or a combination thereof.

20. A cannabinoid nanoparticle prepared by the method of claim 1.
